(19) United States
(12) Patent Application Publication
(10) Pub. No.: US 2021/0262908 A1
(43) Pub. Date: Aug. 26, 2021

(54) SIZE-BASED SEPARATION METHOD FOR HIGHLY CONCENTRATING EXTRACELLULAR VESICLE IN FLUID SAMPLE

(71) Applicant: BIOSOLUTION CO., LTD, Seoul (KR)

(72) Inventors: Junho Kim, Seoul (KR); Ga Eun You, Seoul (KR); Jungsun Lee, Seoul (KR); Song Sun Chang, Seoul (KR)

(21) Appl. No.: 17/253,388

(22) PCT Filed: Jun. 24, 2019
(86) PCT No.: PCT/KR2019/007593

§ 371 Date: Dec. 17, 2020

(30) Foreign Application Priority Data
Jun. 25, 2018 (KR) 10-2018-0072826
Dec. 27, 2018 (KR) 10-2018-0171163

(51) Int. Cl. G01N 1/40; B01D 61/18; B01D 61/14

(52) U.S. Cl. CPC G01N 1/4077; G01N 2001/4088; B01D 61/145; B01D 61/18

(57) ABSTRACT

The present invention relates to a method for concentrating extracellular vesicles in a fluid sample, and specifically, a method for concentrating extracellular vesicles with high efficiency within a short period of time, by using a filter with a pore size of 20 nm to 100 nm and controlling the composition of the fluid sample and the usage of an elution buffer. The method of the present invention for concentrating extracellular vesicles can simplify the process of concentration, reduce the time of concentration, and concentrate extracellular vesicles with high efficiency. That is, the method of the present invention can reduce time and cost for a concentration process and increase concentration efficiency compared to conventional concentration methods, and is thus an economical concentration method suitable for extracellular vesicles.

### TECHNICAL FIELD

The present invention relates to a method for concentrating extracellular vesicles in a fluid sample, and specifically, to a method for concentrating extracellular vesicles with high efficiency within a short period of time by using a filter with an average pore size of 20 nm to 100 nm through a novel method of elution, in which the extracellular vesicles bound to the filter are eluted from a fluid sample containing a surfactant using an elution buffer containing gas.

### BACKGROUND ART

As a new paradigm for treating incurable/intractable diseases, adult stem cell therapy is currently applied in various clinical applications. However, there are risk factors in adult stem cell therapy, such as a risk of zoonosis due to internalization of xenogeneic serum that may occur during an extraction process from a patient or donor or during an in vitro culture process for the proliferation of selected stem cells, a risk of tumor formation that may occur due to stem cell characteristics (e.g., vigorous proliferation, a relatively large cell size, etc.) when stem cells are transplanted into the body, vascular occlusion causing infarcts, etc.

Therefore, methods for avoiding various risk factors or problems that may occur in the treatment directly using live stem cells described above are actively being studied, and as part of this, research results are emerging that extracellular vesicles derived from stem cells may replace therapeutic functions of stem cells (Cell. Mol. Life Sci. (2011) 68: 2667-2688).

Extracellular vesicles are divided into microvesicles, exosomes, etc., and they have a role of exchanging information between cells and function as a bio-maker in connection with cancer cell metastasis, immunity, tissue regeneration, etc.

In particular, separation devices for separating extracellular vesicles, which circulate in blood vessels, from blood have been developed, and an example of these separation devices is a centrifugal separator. The centrifugal separator employs centrifugal force to capture vesicles from nano-sized materials that are formed into pellets. However, since the precipitation rate of particles in the micron size range is very low, centrifugation of these particles takes several minutes to several hours. Due to this problem, much manpower and time are required in the process of dissolving pellets, and a precipitate may be generated due to aggregation of vesicles.

Additionally, in the case of using a centrifugation method, much time is taken, and only about 5% to 25% of the total number of extracellular vesicles contained in the fluid can be separated, while most of the extracellular vesicles (75% to 95%), excluding the separated extracellular vesicles, will be lost. Moreover, the centrifugation method that requires an ultra-high speed centrifuge has disadvantages in that it requires a high-cost device and has a limitation in that thus far, the maximum amount of fluid allowed at a time is 0.6 L (Sci Rep. (2015) August 14; 5: 13103).

### Technical Problem

The present inventors have performed research to develop a method for concentrating extracellular vesicles with high efficiency within a short period of time. As a result, they have found that a large number of extracellular vesicles can be concentrated within a short period of time and the loss of extracellular vesicles can be reduced by using a filter with a specific pore size, controlling the composition of a fluid sample, and controlling the usage of an elution buffer, thereby completing the present invention.

### Technical Solution

An object of the present invention is to provide a method for concentrating extracellular vesicles in a fluid sample with high efficiency and within a short period of time, and specifically, a method for concentrating extracellular vesicles with high efficiency by using a filter with an average pore size of 20 nm to 100 nm and controlling the composition of the fluid sample and the usage of an elution buffer.

### Advantageous Effects

When the method of the present invention for concentrating extracellular vesicles is used, extracellular vesicles can be concentrated with high efficiency while simplifying the concentration process and reducing the time required for the concentration. The method of the present invention can reduce the time and cost for concentration while increasing concentration efficiency compared to conventional concentration methods, and it proposes a cost-effective concentration method suitable for extracellular vesicles. In particular, the characteristics of the filter, fluid sample, and elution buffer used in the present invention are useful in that they are not limited to a specific device but can be applied to various known devices.

### BRIEF DESCRIPTON OF THE DRAWINGS

FIG. 1A-B shows a concentrating pipette tip used in the present invention.

FIG. 2 shows a graph illustrating the total volume of a culture medium, which is separated from extracellular vesicles by a method using a 50 nm filter.

FIG. 3 shows a graph illustrating the total volume of a culture medium, which is separated from extracellular vesicles by a method using an ultrafilter (≤20 nm).

FIG. 4 shows a graph illustrating the number of particles in concentrated extracellular vesicles per unit volume according to the type of filter. Fold represents a value obtained by confirming the concentrated ratio compared to a culture medium.

FIG. 5 shows a graph illustrating the total number of particles in extracellular vesicles obtained according to the type of filter.

FIG. 6 shows a graph illustrating the relative content of impurities according to the type of elution buffer.

FIG. 7 shows a graph illustrating the measurement of the size of impurities according to the type of elution buffer.

FIG. 8 shows a graph illustrating the recovery efficiency of extracellular vesicles according to the number of use of an elution buffer (1 time and 3 times).

FIG. 9 shows a graph illustrating both the recovery efficiency of concentrated extracellular vesicles (concentrated EVs) according to the filter size (<20 nm, 50 nm, 100 nm, and 200 nm), in which the number of use of an elution buffer is fixed (3 times); and the removal efficiency of extracellular vesicles (filtrated EVs), which are not concentrated and discharged by passing through a filter.

FIG. 10 shows a graph comparing the recovery efficiency between the extracellular vesicles concentrated by the concentration method of the present invention and the extracellular vesicles concentrated by a concentration method using ExoQuick.

FIG. 11 shows images confirming the shapes of the extracellular vesicles concentrated by the concentration method of the present invention and the extracellular vesicles concentrated by a concentration method using ExoQuick.

FIG. 12 shows graphs comparing the size between the extracellular vesicles concentrated by the concentration method of the present invention and the extracellular vesicles concentrated by a concentration method using ExoQuick.

FIG. 13 shows a graph illustrating the costs required for the concentration of extracellular vesicles when the extracellular vesicles are concentrated by the concentration method of the present invention and when the extracellular vesicles are concentrated by a concentration method using ExoQuick.

FIG. 14 shows a graph illustrating the recovery efficiency of extracellular vesicles according to the concentration of a surfactant contained in a fluid sample (0%, 0.05%, 1%, 5%, and 10%).

### BEST MODE FOR CARRYING OUT THE INVENTION

A first aspect of the present invention provides a method for concentrating extracellular vesicles in a fluid sample, wherein the method, which uses a device comprising (i) a container for accommodating a fluid sample; (ii) a concentrating pipette tip, which comprises a filter with an average pore size of 20 nm to 100 nm, an inlet, and an outlet; and (iii) a concentration unit, which is configured to aspirate the fluid sample through the concentrating pipette tip and to collect the concentrated extracellular vesicles from the concentrating pipette tip, comprises:
aspirating the fluid sample contained in the container of (i) through the concentrating pipette tip; and
eluting the extracellular vesicles captured in the filter of the concentrating pipette tip.

Hereinafter, the present invention is described in detail.

### DETAILED DESCRIPTION

One alternative method to the centrifugation method for separating extracellular vesicles is a method for concentrating extracellular vesicles using a polymer called polyethyleneglycol (PEG). This method utilizes the property of PEG to lower the solubility of EVs and induce EVs to precipitate easily. As the reaction time between PEG and extracellular vesicles increases, a large number of extracellular vesicles precipitate, and a reaction time of 4 hours to 24 hours is usually required. ExoQuick is known as a representative product using the elution method by PEG. The method of the present invention for separating extracellular vesicles is effective in terms of productivity because the amount of separation is nearly twice as high, and the cost is 20 times or more cheaper compared to that of ExoQuick.

In addition to the precipitation method, a method for separating extracellular vesicles commonly used is a method using a filter. The filtration method is a method of filtering extracellular vesicles using a filter having a pore size smaller than or similar to the size of the extracellular vesicles. Since extracellular vesicles are filtered by a filter and other proteins are discharged, the separation speed can be adjusted according to the speed of the fluid, and the separation time is fast because time for reaction is not required. However, it has been thought that there is a limitation in that the final recovery rate is not high due to the high proportion of extracellular vesicles that bind to or pass through the filter. The filtration method of the present invention is characterized in that the final separation efficiency is improved by lowering the ratio of the extracellular vesicles that bind to or pass through a filter, by adjusting the filter size and the constituents of an elution buffer while using a conventional filtration method, thus retaining the advantages of the conventional filtration method.

In order to solve the conventional problems, the present inventors performed studies to develop a method for concentrating extracellular vesicles at low cost within a short period of time. As a result, they have discovered that in the case where (i) a filter with an average pore size of 20 nm to 100 nm is used, (ii) elution is performed by repeatedly using an elution buffer which contains a Tris elution buffer, a PBS elution buffer, or a bubble-forming gas, and (iii) an additional surfactant is included in a fluid sample containing extracellular vesicles, extracellular vesicles can be concentrated with high efficiency while reducing the time and cost compared to the conventional concentration method. The present invention is based on this discovery.

Specifically, the concentration method of the present invention is a method for concentrating extracellular vesicles using a filter and an elution buffer. The concentration device used in the present invention is a device, which comprises (i) a container for accommodating a fluid sample; (ii) a concentrating pipette tip, which comprises a filter with an average pore size of 20 nm to 100 nm, an inlet, and an outlet; and (iii) a concentration unit, which is configured to aspirate the fluid sample through the concentrating pipette tip and to collect the concentrated extracellular vesicles from the concentrating pipette tip.

In the device of the present invention, the concentrating pipette tip may be one which separately includes an elution port. That is, the concentrating pipette tip may be one in which the inlet included in the concentrating pipette tip simultaneously performs aspiration and elution of a sample. However, when an elution port is additionally included in the concentrating pipette tip, a fluid sample and an eluent can be aspirated through mutually different parts.

The device used for the concentration method of the present invention will be described in detail as follows.

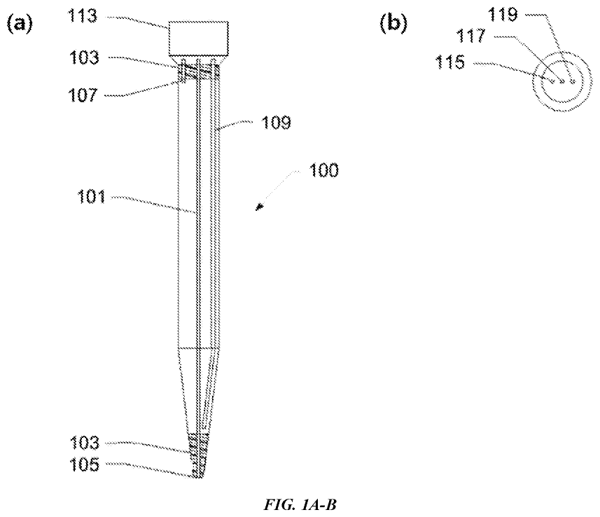

FIG. 1(a) shows a concentrating pipette tip 100, and specifically shows a concentrating pipette tip 100 which includes an inlet 105 and a filter 101. Additionally, the concentrating pipette tip 100 includes a filter 101, a permeate purge 107, and a potting 103 of a permeate draw 109. The connection part 113 shown in the concentrating pipette tip 100 allows the concentrating pipette tip 100 to be connected to a concentration unit for operation of the concentrating pipette tip 100.

FIG. 1(b) shows the connection part 113 which includes three ports. The connection part 113 consists of a first port 115 connected to the permeate purge 107, a second port 117 connected to the filter 101, and a third port 119 connected to the permeate draw 109. The first port 115, the second port 117, and the third port 119 are connected to the concentration unit through the connection part 113, and the concentrating pipette tip 100 is connected to the concentration unit. A fluid sample is aspirated into the lower inlet 105 and passes through the porous surface of the filter 101 using a pump connected to the permeate draw 109 through the third port 119. The filter 101 or another thin film filter is a dry hydrophilic filter, a glycerin-filled hydrophilic filter, or another type of filter that initially allows air to pass therethrough, and when the filter comes in contact with liquid, it allows the liquid to pass through. That is, air is aspirated into the lower inlet 105 and passes through the porous surface of the filter 101, until the fluid sample is aspirated through the lower inlet 105 and comes in contact with the filter 101 and passes through the porous surface.

The concentration system used in the present invention is as follows.

All of the tubes, in which a disposable concentrating pipette tip 100 is connected to the concentration unit, are connected at a single connection point located on top of the concentrating pipette tip 100. The concentrating pipette tip 100 functions along with a system which includes a concentration unit and a fluid sample. The concentration unit operates by immersing the inlet 105 of the concentrating pipette tip 100 into a fluid sample contained in a suitable sample container. Then, the fluid sample is aspirated into the concentrating pipette tip 100 and comes into contact with the filter 101. While the liquid passes through the filter 101, particles which are similar to or larger than the pore size of the filter 101 are captured and retained in the filter 101. After all of the samples pass through the filter 101, the fluid is removed and only the captured sample remains. Then, the lower inlet 105 of the concentrating pipette tip 100 is immersed into an appropriate container, and the captured materials are eluted using an elution buffer, and thereby concentrated extracellular vesicles are obtained.

The method of the present invention for concentrating extracellular vesicles includes aspirating a fluid sample through the concentrating pipette tip and eluting extracellular vesicles captured in the filter of the concentrating pipette tip.

The fluid sample may be one which further includes a surfactant in an amount of 0.05 wt % to 5 wt %. Specifically, the fluid sample may be one which includes a surfactant in an amount of 0.1 wt % to 4 wt %, 0.3 wt % to 3 wt %, 0.5 wt % to 2 wt %, and 0.75 wt % to 1.5 wt %, and more specifically 1 wt %. When a surfactant is further included in the fluid sample as described above, the extracellular vesicles bound to the filter are easily separated and induced to be concentrated, thereby allowing improvement of the separation efficiency of extracellular vesicles.

The surfactant of the present invention may be poloxamer, polysorbate, or a combination thereof. For example, the surfactant is characterized in that it is selected from the group consisting of poloxamer 188, polysorbate 20 (Tween 20), polysorbate 40 (Tween 40), polysorbate 60 (Tween 60), polysorbate 80 (Tween 80), and/or a combination thereof. In the examples of the present invention, Tween 20 was used as a representative surfactant.

The elution may be performed using an elution buffer which includes a Tris elution buffer or a PBS elution buffer. Additionally, the elution may be performed 1 time to 5 times.

Additionally, the elution buffer may further include bubble-forming gas, and more specifically may include carbon dioxide, nitrogen, argon, air, liquefied petroleum gas, or a combination thereof.

In the case where a fluid sample including a surfactant is mixed with an elution buffer including bubble-forming gas, the gas reacts with the surfactant and generates microscopic droplets, thereby increasing the volume of the filter it encounters, and as a result, induces the elution buffer to elute the extracellular vesicles in the filter without empty space.

In the method of the present invention for concentrating extracellular vesicles, the average pore size of the filter 101 used for the capture of extracellular vesicles may be 20 nm to 100 nm, and more specifically 30 nm to 80 nm, 40 nm to 60 nm, or 50 nm. These are appropriate sizes to more effectively capture extracellular vesicles.

The size of extracellular vesicles is known to be 20 nm to 1μm. When a filter with an average pore size of less than 20 nm is used, there is difficulty in that concentration of extracellular vesicles may not proceed because the time required for liquids or proteins other than extracellular vesicles to escape the filter is prolonged or they may clog the filter. Additionally, when the average pore size of the filter is larger than the size of extracellular vesicles, there is a problem in that the rate at which extracellular vesicles are lost increases. In particular, when the average pore size of the filter is 100 nm or larger, there is a problem in that among the extracellular vesicles, the proportion of small extracellular vesicles (exosomes) with a size of about 20 nm to about 100 nm decreases.

Therefore, for efficient recovery in which the loss rate of extracellular vesicles is reduced while the efficiency of removing liquids and proteins is increased, filters with an optimal pore size should be used. The present invention is characterized in that a filter with an average pore size of 20 nm to 100 nm is selected as an efficient filter which is capable of maximally increasing the total amount of the extracellular vesicles to be recovered.

That is, in the present invention, the conditions with respect to the average pore size of a filter, changes in the composition of a fluid sample, the composition of an elution buffer, the number of elution, etc. suitable for concentrating extracellular vesicles were determined, and thereby the combination for the optimal concentration method was discovered.

Additionally, the surface area of the filter of the present invention may be 5 cm² to 20 m², but the surface area is not limited thereto. Any filter which has an average pore size of 20 nm to 100 nm is applicable to the concentration method of the present invention regardless of the surface area of the filter.

In the concentration method of the present invention, as a solution for eluting extracellular vesicles captured in the filter 101, a Tris elution buffer or a PBS elution buffer may be used. In order to use an eluant, it is important to select the eluant after checking the concentration of small impurities (nanoparticles) in the eluant and to then minimize the amount of the eluant to be mixed with extracellular vesicles to be concentrated later.

In addition, the number of uses of the eluant is important to increase the elution efficiency of extracellular vesicles. In the present invention, it was confirmed that while using the filter with the above-described average pore sizes, extracellular vesicles can be separated with optimal efficiency when the number of uses of the eluent (i.e., the number of elutions) is 1 time to 5 times. When the number of elutions exceeds 5 times, the number of extracellular vesicles to be separated may increase, but there may be a problem in that the concentration of extracellular vesicles may be diluted or that the shape of extracellular vesicles may be destroyed.

The extracellular vesicles of the present invention are vesicles which are produced in cells and secreted outside the cells, and they refer to a structure in the form of particles, in which various biomolecules, such as proteins having various functions, which are released (secreted) from cells to an extracellular environment, (e.g., various kinds of growth factors, chemokines, cytokines, transcription factors, RNAs (mRNA, miRNA, etc.), lipids, etc.) are enclosed in a cell membrane of a lipid bilayer, which is identical to that of the cell from which these various biomolecules are derived. The extracellular vesicles include exosomes, microvesicles, microparticles, etc., but are not limited thereto.

The concentrated extracellular vesicles can be applied to analysis methods that are commonly used in the art. For example, the analysis methods may include immunoassay, polymerase chain reaction (PCR), electrochemical analysis, microarray, flow cytometry, biosensors, labs-on-a-chip, rapid growth based detection, etc., but the analysis methods are not limited thereto.

In particular, the major technical features in the concentration method of the present invention are the type of filter, the type of elution buffer, and the number of use of elution buffer. All of the embodiments of various concentration methods using filters available in the art are included in the scope of the present invention.

The concentration method of the present invention may further include a step of separating extracellular vesicles. The extracellular vesicles eluted by the method of the present invention may be used in addition to various known methods for separating extracellular vesicles, but the methods are not limited thereto. For example, the method for separating extracellular vesicles may include a polymer-based separation method, a centrifugation-based separation method, a concentration-based separation method, and a filtration-based separation method, but the method for separating extracellular vesicles is not limited thereto.

### EXAMPLES

Hereinafter, the present invention will be described in more detail with reference to the following Examples. However, these Examples are for illustrative purposes only, and the scope of the invention is not limited by these Examples.

### Example 1

### Cell Culture and Acquisition of Culture Medium

In this study, a culture medium was used in which adipose stem cells were cultured. Specifically, adipose stem cells were cultured at a confluence of about 80% to 90% in culture dishes, and then the medium was replaced with a MEM-alpha medium (Gibco) containing a 10% serum replacement (Gibco). Two days thereafter, a raw culture medium of the adipose stem cells was collected in each tube, and the dead cells were settled by centrifugation (3,000 g, 20 minutes), and the supernatant was separated. The obtained supernatant was used in the following Examples, and the supernatant is hereinafter referred to as the “culture medium”.

### Example 2

### Analysis of Concentration Volume Over Time

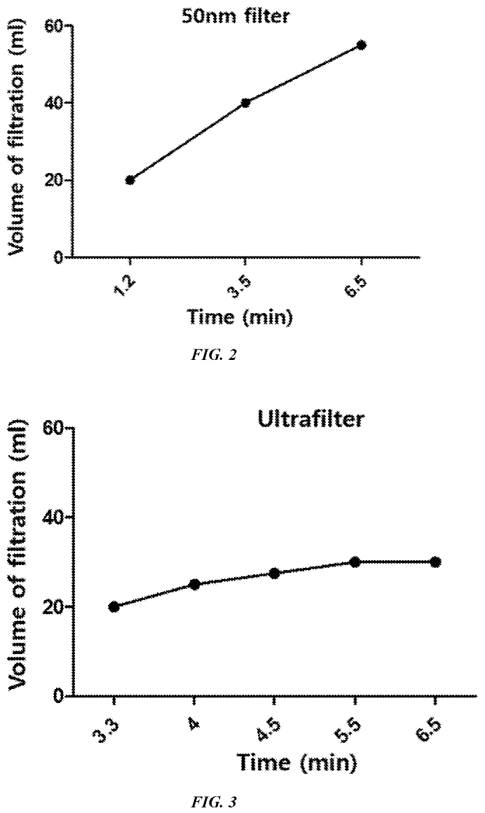

While varying the pore size of a filter, the total volume of a culture medium which passed through the filter was measured over time, and the results are shown in FIGS. 2 and 3.

When a filter having a pore size of 50 nm was used, clogging of the filter occurred less frequently, and the total volume of the culture medium passing through the filter steadily increased as time passed, and it was possible to perform the concentration at an average rate of 55 mL/6.5 minutes. In addition, it was confirmed that the total amount of the culture medium that can be finally concentrated was about 60 mL (FIG. 2). In contrast, when an ultrafilter was used, the maximum amount of the culture medium that can be concentrated was only about 30 mL. Therefore, the concentration volume did not increase beyond a certain level even after time passed, and finally, it was calculated to be concentrated at an average rate of 27 mL/6.5 minutes (FIG. 3).

That is, when comparing the overall concentration rate and concentration amount, it was found that when a filter with a pore size of 50 nm was used, the efficiency was about almost twice as high. In addition, in the case of an ultrafilter (<20 nm), it was confirmed that the filter was easily clogged.

### Example 3

### Confirmation of Number of Extracellular Vesicles Contained Per Unit Volume (Confirmation of Concentration Level)

30 mL of a culture medium was aspirated through a 50 nm filter or an ultrafilter, and then eluted once with a Tris elution buffer. The concentration of extracellular vesicles in each eluted concentrate was measured with a Nanoparticle tracking analysis device (Malvern, LM10).

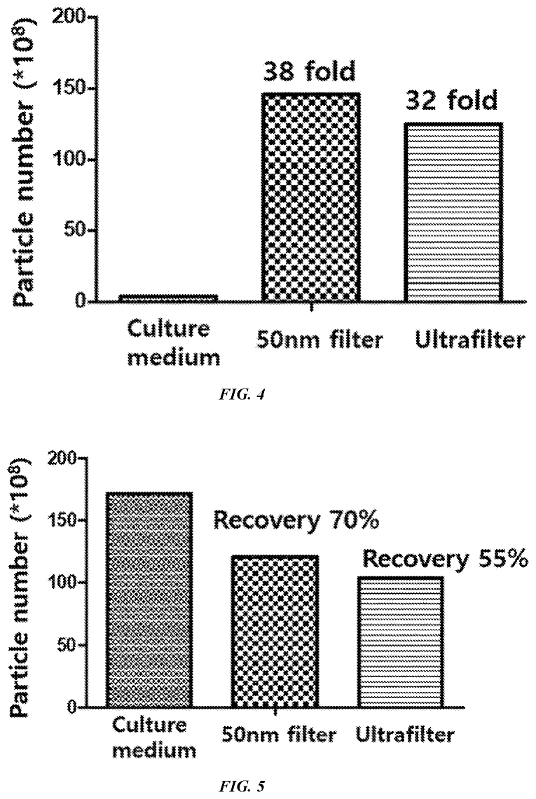

As shown in FIG. 4, it was confirmed that the number of extracellular vesicles per single volume was about 38 times that of the culture medium before concentration. When 30 mL of a culture medium was concentrated using an ultrafilter, it was confirmed that the number of extracellular vesicles per single volume was about 32 times that of the culture medium. From these results, it was confirmed that when a filter having a pore size of 50 nm is used, extracellular vesicles can be concentrated at a higher rate compared to when an ultrafilter was used.

### Example 4

### Confirmation of Total Number of Extracellular Vesicles Recovered

10 mL of a culture medium was aspirated through a 50 nm filter or an ultrafilter, and then eluted once with a Tris elution buffer. The concentration of extracellular vesicles in each eluted concentrate was measured with a Nanoparticle tracking analysis device (Malvern, LM10), and the results are shown in FIG. 6. Recovery efficiency represents the ratio of the separated extracellular vesicles to the total extracellular vesicles contained in the culture medium.

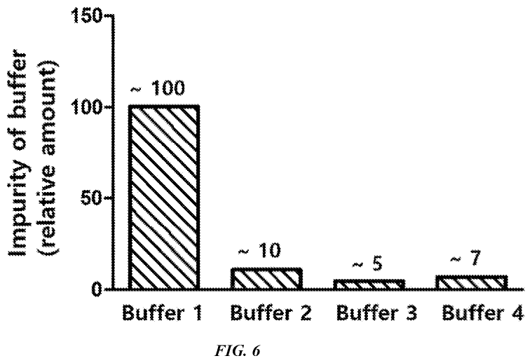

As shown in FIG. 5, while 70% of the extracellular vesicles present in the existing culture medium were recovered when a 50 nm filter was used, when an ultrafilter was used, a recovery rate of about 55% was shown. Therefore, it was confirmed that the recovery rate when an ultrafilter was used was significantly lower compared to when a filter with a size of 50 nm was used.

### Example 5

### Comparison of Impurities According to Elution Buffer

Liquid materials are highly likely to contain impurities with a size similar to that of extracellular vesicles. Therefore, the presence of impurities in the elution buffer was tested for confirmation purpose. The relative amount and size of the impurities in each elution buffer were measured with a nanoparticle tracking analysis device (Malvern, LM10). Buffers 1 and 3 are PBS elution buffers, and Buffers 2 and 4 are Tris elution buffers.

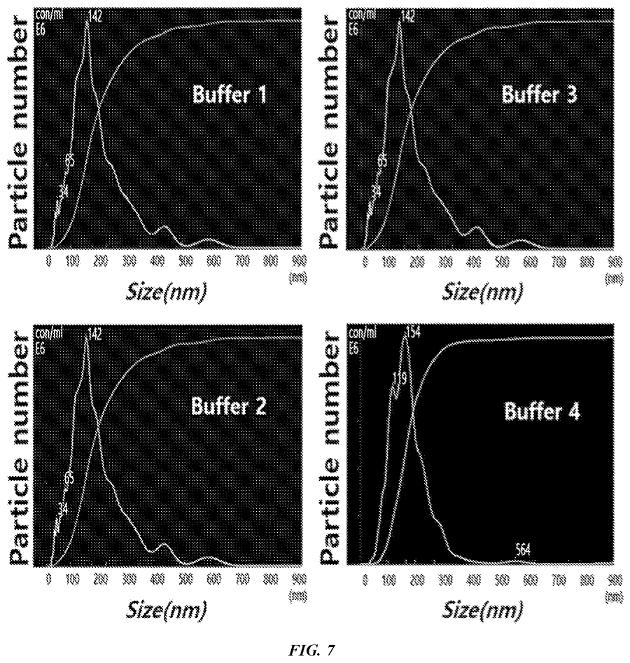

As shown in FIG. 6, even in the same elution buffer, the degree of impurities contained varied. Additionally, as shown in FIG. 7, since the impurities have various sizes ranging from 20 nm (i.e., nano size) to 1 μm, it is highly likely to be difficult to distinguish these impurities from extracellular vesicles. Therefore, an elution buffer containing a lesser amount of impurities was selected and subjected to a test.

### Example 6

### Confirmation of Increase in Total Yield According to Number of Use of Elution Buffer

For Group 1, in which 10 mL of a culture medium was aspirated through a 50 nm filter and then eluted once with a Tris elution buffer and extracellular vesicles bound to the filter were eluted; and for Group 2, in which 10 mL of the culture medium was aspirated through a 50 nm filter and then eluted 3 times with a Tris elution buffer and extracellular vesicles bound to the filter were eluted, extracellular vesicles were measured with a nanoparticle tracking analysis device (Malvern, LM10) and their recovery rates were compared. The recovery rate represents the ratio of the separated extracellular vesicles to the total extracellular vesicles contained in the culture medium.

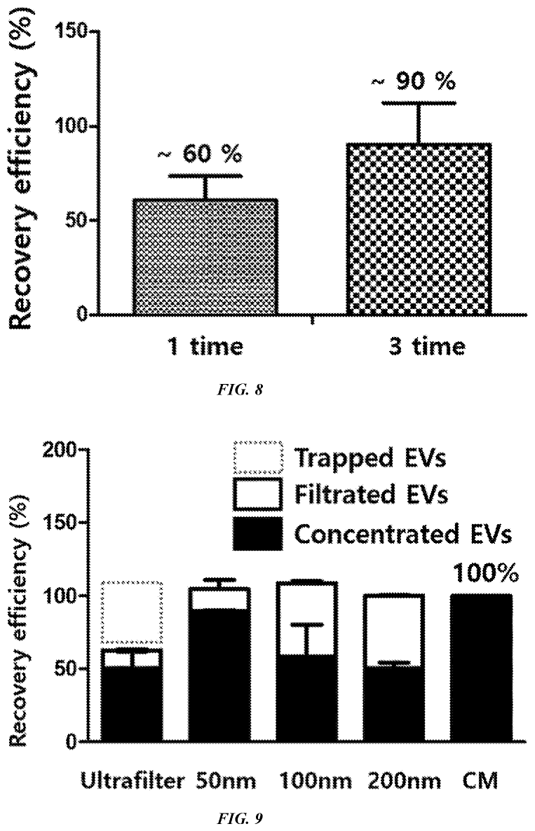

As shown in FIG. 8, the recovery rate was 60% in the group where the elution buffer was used once and 90% in the group where the elution buffer was used 3 times. In other words, the recovery rate was increased by increasing the number of elutions to 3 times.

### Example 7

### Comparison of Total Yield According to Size of Various Filters

The total yield according to the filter size was compared using an ultrafilter (<20 nm), a 50 nm filter, a 100 nm filter, and a 200 nm filter.

10 mL of a culture medium was aspirated through an ultrafilter, a 50 nm filter, a 100 nm filter, and a 200 nm filter, and then the culture medium was treated 3 times with a Tris elution buffer to elute the extracellular vesicles bound to the filter. In addition to the measurement of the concentrated extracellular vesicles, the extracellular vesicles which passed through the filter and which were discharged without being concentrated were also compared together. The extracellular vesicles in each group were measured with a Nanoparticle tracking analysis device (Malvern, LM10), and their recovery rates were compared.

As shown in FIG. 9, the 50 nm filter showed a high recovery rate close to about 90% (black graph, concentrated EVs), but other filters (i.e., the ultrafilter, the 100 nm filter, and the 200 nm filter) showed low recovery rates of about 50%.

From the results above, it was found that in the cases of the 100 nm filter and the 200 nm filter, many extracellular vesicles were discharged (white graph, filtrated EVs). In addition, in the case of the ultrafilter, even when the numbers of the concentrated extracellular vesicles and the amount of the discharged extracellular vesicles were combined, this combined number was very insufficient compared to the total number of extracellular vesicles present in the existing culture medium. This indicates that extracellular vesicles were trapped inside the ultrafilter and could not be eluted or passed through the filter (dotted line graph, trapped EVs).

### Example 8

### Comparison of Total Yield with ExoQuick

The separation efficiency was compared between the conventionally known concentration method of using ExoQuick (product name: ExoQuick-TC™), which is a liquid mixed with PEG, and the concentration method of the present invention.

In the case of the concentration method of the present invention, 10 mL of a culture medium was aspirated through a 50 nm filter, and then eluted using a Tris elution buffer 3 times. Additionally, 10 mL of the culture medium was mixed with 2 mL of ExoQuick, and then extracellular vesicles were allowed to elute from the culture medium for 24 hours. The eluted extracellular vesicles were separated by settling in a centrifuge (1,500 g) for 30 minutes. The separated extracellular vesicles in each group were analyzed with a Nanoparticle tracking analysis device (Malvern, LM10).

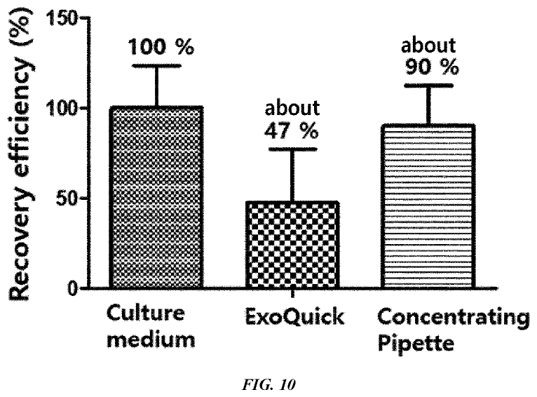

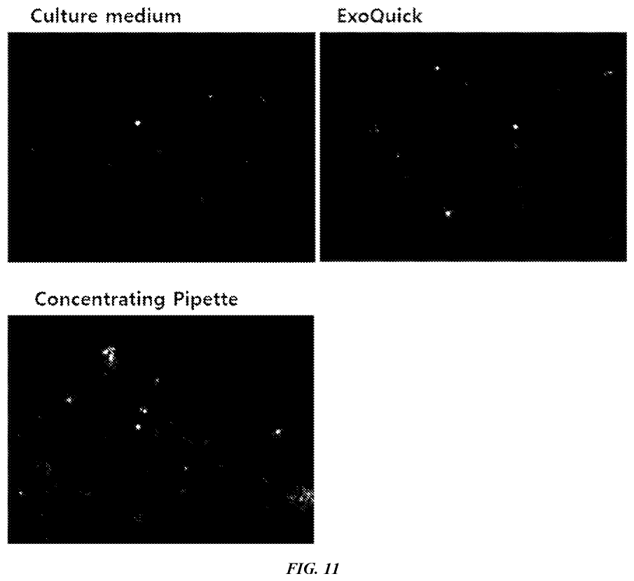

As shown in FIG. 10, in the case of the concentration method of the present invention where elution was performed 3 times using a 50 nm filter and an elution buffer, the extracellular vesicles were separated with an efficiency of 90.1%. In contrast, it was confirmed that the recovery rate was only 47.4% when ExoQuick was used.

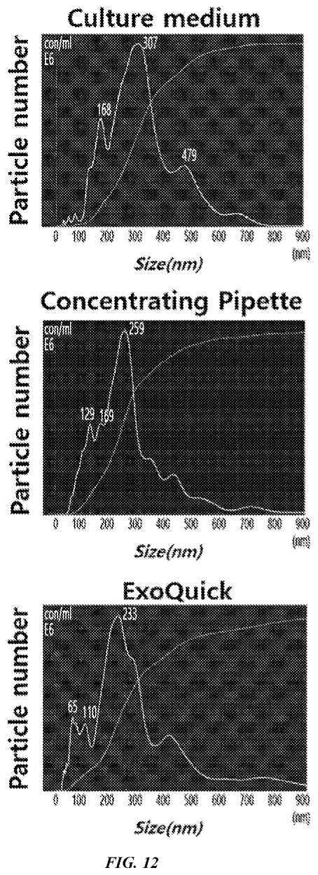

Additionally, in the images of extracellular vesicles measured by Nanosight, it was found that the extracellular vesicles separated by the concentration method of the present invention were identical to the extracellular vesicles separated using a culture medium and Exoquick (FIG. 11). Further, even in the measurement results of the size of extracellular vesicles, it was found that the size of the extracellular vesicles separated by the concentration method of the present invention was similar to that of the extracellular vesicles separated using a culture medium and ExoQuick (FIG. 12). These results indicate that the concentrating pipette of the present invention increases the separation yield compared to the conventional method, but also does not cause any deformation to extracellular vesicles.

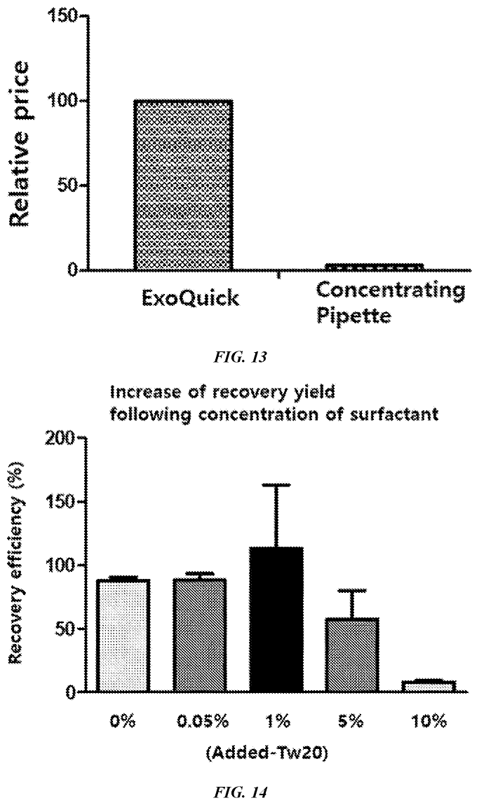

ExoQuick requires an ExoQuick kit as a consumable and centrifuge equipment. In the case of a concentrating pipette, a filter and an elution buffer are required as consumables and a pump is required as equipment. As shown in FIG. 13, comparing in terms of the cost of consumables, it was found that the cost for concentrating extracellular vesicles in 60 mL of a culture medium was about KRW 600,000 for ExoQuick, and that the cost for the concentrated pipette was less than about KRW 40,000. It was confirmed that the cost can be reduced by at least 15 times when the concentrating pipette of the present invention is used compared to when ExoQuick is used.

### Example 9

### Comparison of Yields of Extracellular Vesicles According to Surfactant Concentration

The total yield was compared according to the concentration of a surfactant contained in a fluid sample (0%, 0.05%, 1%, 5%, and 10%).

Tween 20 was prepared into 0%, 0.05%, 1%, 5%, and 10% in 10 mL of a culture medium. Each culture medium was aspirated through a 50 nm filter and then treated 3 times with a Tris elution buffer, and thereby extracellular vesicles bound to the filter were eluted. The eluted extracellular vesicles were each measured with a Nanoparticle tracking analysis device (Malvern, LM10), and their recovery rates were compared.

As shown in FIG. 14, it was confirmed that when 1% of a surfactant was contained in the fluid sample, the yield of extracellular vesicles was the highest. Additionally, it was found that when 5% and 10% of a surfactant was contained in the fluid sample, the concentration was not suitable for use because the surfactant dissolved and destroyed extracellular vesicles. That is, although the method of separating a fluid sample by further containing a surfactant can increase the separation efficiency of extracellular vesicles, but it was found that the appropriate concentration of a surfactant for the separation efficiency of extracellular vesicles was about 1%.

From the foregoing, one of ordinary skill in the art to which the present invention pertains will be able to understand that the present invention may be embodied in other specific forms without modifying the technical concepts or essential characteristics of the present invention. In this regard, the exemplary embodiments disclosed herein are only for illustrative purposes and should not be construed as limiting the scope of the present invention. On the contrary, the present invention is intended to cover not only the exemplary embodiments but also various alternatives, modifications, equivalents, and other embodiments that may be included within the spirit and scope of the present invention as defined by the appended claims.

1. A method for concentrating extracellular vesicles in a fluid sample, wherein the method, which uses a device comprising (i) a container for accommodating a fluid sample; (ii) a concentrating pipette tip, which comprises a filter with an average pore size of 20 nm to 100 nm, an inlet, and an outlet; and (iii) a concentration unit, which is configured to aspirate the fluid sample through the concentrating pipette tip and collect the concentrated extracellular vesicles from the concentrating pipette tip, comprises:
aspirating the fluid sample contained in the container of (i) through the concentrating pipette tip; and
eluting the extracellular vesicles captured in the filter of the concentrating pipette tip.

2. The method of claim 1, wherein the concentrating pipette tip comprises an elution port.

3. The method of claim 1, wherein the elution is performed using an elution buffer comprising a Tris elution buffer or a PBS elution buffer, and the elution is performed 1 to 5 times.

4. The method of claim 3, wherein the elution buffer comprises carbon dioxide, nitrogen, argon, air, liquefied petroleum gas, or a combination thereof.

5. The method of claim 1, wherein the surface area of the filter is in a range of 5 cm² to 20 m².

6. The method of claim 1, wherein the fluid sample further comprises a surfactant in an amount of 0.05 wt % to 5 wt %.

7. The method of claim 1, further comprising a step of separating the extracellular vesicles.
